(19) United States
(12) Patent Application Publication
(10) Pub. No.: US 2025/0195720 A1
(43) Pub. Date: Jun. 19, 2025

(54) SILICONE OIL IN WATER INJECTABLE FILLER COMPOSITION USED AS A SCAFFOLD FOR COLLAGEN GROWTH

(71) Applicant: Mark Mezzancello, palm beach gardens, FL (US)

(72) Inventor: Mark Mezzancello, palm beach gardens, FL (US)

(21) Appl. No.: 18/544,429

(22) Filed: Dec. 19, 2023

(51) Int. Cl. A61L 27/26; A61L 27/54; C08L 1/28; C08L 83/04

(52) U.S. Cl. CPC A61L 27/26; A61L 27/54; C08L 1/286; C08L 83/04; A61L 2300/402; A61L 2300/428; A61L 2400/06; A61L 2430/34

(57) ABSTRACT

A silicone oil in water composition that is an injectable filler that is useful as a scaffold for collagen growth in a human. The injectable filler composition comprises of 1.0% to 80.0% of a silicone oil having a viscosity of 10000 centistokes, 20.0% to 80.0% of a water, and 1.5% to 3.0% of a thickening agent, wherein the filler composition is an oil in water emulsion, the silicone oil droplets will have an average diameter from 30 microns to 2000 microns.

### BACKGROUND

The present invention is directed to a silicone oil in water composition that is an injectable filler that is useful as a scaffold for collagen growth in a human.

The present invention was developed to provide a composition that can be applied to a human subcutaneously to augment the soft tissue of the human.

The present invention was developed to allow people who suffer from a sensitivity to bovine collagen, a common filler used to augment soft tissue, to receive a filler composition that does not contain bovine collagen. It is estimated that 2 to 4 percent of the population have a pre-existing sensitivity to bovine collage and that 1 percent react to collagen.¹

The present invention was also developed to stimulate collagen production in a human by up to 250% more than it is being presently produced after a human receives an injectable filler that is made of a composition that will augment soft tissue.

The present invention can be ideally used to enhance the genital soft tissues of a human. Yet, it is foreseen that the present invention can also be used to augment other parts of the body for either aesthetic purposes or to correct congenital or acquired conditions. ¹ Available at https://www.aaaai.org/allergist-resources/ask-the-expert/answers/2022/bovine#: ˜: text=There%20is%20some%20evidence%20that,have%20been%20reported%20 (5)., American Academy of Allergy Asthma & Immunology, titled Bovine Collagen Allergy, Feb. 23, 2022, seen on Nov. 26. 2023.

The present invention addresses the need of having an injectable filler composition that can be applied to humans who suffer from a collagen sensitivity that will stimulate collagen production by at least 250%.

### SUMMARY

The present invention is directed to an injectable composition that can be applied to humans who suffer from a collagen sensitivity that will stimulate collagen production by at least 250%.

The injectable filler composition comprises of 1% to 80% of a silicone oil having a viscosity of 10000 centistokes, 20% to 99% of water, and 1.5% to 3.00% of a thickening agent, wherein the filler composition is an oil in water emulsion, the silicone oil droplets will have an average diameter from 30 microns to 2000 microns.

The thickening agent will be a composition that comprises of a carboxymethyl cellulose, a 1,4 butanediol diglycidyl ether, a polysorbate, a methyl paraben, a lidocaine, and a silica powder. The present invention uses a highly purified silicone and sterile water.

Vitamin C, ascorbic acid is used in the composition, for ascorbic acid is a critical enzyme that is used to produce a type 1 or a type 2 collagen. In a test conducted using the present composition, the production of collagen was increased by 297%.

Silica is a naturally occurring mineral in which the backbone structure consists of alternating silicon and oxygen atoms (SiO2). Silica is used in the present invention to increase the viscosity of the composition and to stimulate collagen production.

An object of the present invention is to provide an injectable filler that can be applied to humans who suffer from a bovine collagen sensitivity.

Another object of the present invention is to provide an injectable filler that will promote the production of collagen by at least 250%.

Yet another object of the present invention is to provide an injectable filler that can be safely applied to a human subcutaneously for the enhancement of soft tissue.

Yet still another object of the present invention is to provide an injectable filler that can be used to augment the size the penis of a man.

Yet still further object of the present invention is to provide an injectable filler that can be used to aesthetically address congenital defects or acquired defects of a human.

### DESCRIPTION

All percentages and ratios used herein, unless otherwise indicated, are by weight.

As used herein, use of the term biodegradable to describe a material that is degraded when implanted on a human.

The term viscosity is used herein in its standard sense as a measure of the resistance of fluid to gradual deformation by shear stress.

The term emulsion as used herein means a mixture of two or more liquids that are not normally immiscible. An example of an emulsion is an oil in water emulsion in which the oil phase is dispersed in the continuous water phase. In the present invention the composition is an oil in water emulsion wherein the oil phase is a silicone oil.

The invention can be embodied in other specific forms without departing from the spirit or essential characteristics thereof. The foregoing embodiments are to be considered in all respects illustrative rather than limiting on the invention described herein. In the various embodiments of the present invention, such as for example, the compositions and methods of the present invention, where the term comprises is used, it is also contemplated in other embodiments that the present; invention consists essentially of, or consists of, the embodiments. Further, it should be understood that the order of steps or order for performing certain actions is immaterial so long as the invention remains operable. Moreover, two or more steps or actions can be conducted simultaneously in some instances.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

Furthermore, it should be recognized that in certain instances a composition can be described as being composed of the components prior to mixing, because upon mixing certain components can further react or be transformed into additional materials.

The present invention is an injectable filler composition that can be applied to humans who suffer from a collagen sensitivity that will stimulate collagen production. The injectable filler composition comprises 1.0% to 80.0% of a silicone oil having a viscosity of 10,000 centistokes. 20.0% to 99.0% of a water. And, 1.5% to 3.0% of a thickening agent, wherein the injectable filler composition is an oil in water emulsion, the silicone oil droplets will have an average diameter from 30 microns to 2000 microns.

In preferred embodiments of the present invention, the thickening agent is a composition comprised of from about 1.5% to about 3% of a carboxymethyl cellulose. From about 0.05% to about 1.0% of a 1,4 butanediol diglycidyl ether. From about 0.3% to about 0.6% of an ascorbic acid. From 0.2% to about 0.6% of a polysorbate. From about 0.1% to about 0.2% of a methyl paraben. About 0.3% lidocaine. From about 0.01% to about 0.5% silica. About a 50% lactated Ringer's Solution. From about 43.8% to about 47.54% sterile water for injection.

In all embodiments of the present invention, the thickening agent is biodegradable when implanted subcutaneously in a human to provide a temporary scaffold for collagen growth between silicone oil droplets.

An advantage of the present invention is that it provides an injectable filler that can be applied to humans who suffer from a bovine collagen sensitivity.

Another advantage of the present invention is that it provides an injectable filler that promotes the production of collagen by at least 250%.

Yet another advantage of the present invention is that it provides an injectable filler that can be safely applied to a human subcutaneously for the enhancement of soft tissue.

Yet still another advantage of the present invention is that it provides an injectable filler that can be used to augment the size of the penis of a man.

Yet still further advantage of the present invention is that it provides an injectable filler that can be used to aesthetically address congenital defects or acquired defects of a human. The above composition and method variants of this example demonstrate that the

compositions and methods of the present invention are useful for stimulating collagen production for male enhancement. The compositions and methods of the present invention are also useful for stimulating collagen production for face or body skin wrinkle reduction, scar repair, and breast enhancement

While the invention has been described in detail and with reference to specific examples thereof, it will be apparent to one skilled in the art that various changes and modifications can be made therein without departing from the spirit and scope thereof.

What is claimed is:

1. An injectable filler composition that can be applied to humans who suffer from a collagen sensitivity that will stimulate collagen production, the injectable filler composition comprises:
from about 1.0% to about 80.0% of a silicone oil having a viscosity of 10,000 centistokes;
from about 20.0% to about 99.0% of a water; and
from about 1.5% to about 3.0% of a thickening agent, wherein the injectable filler composition is an oil in water emulsion, the silicone oil droplets will have an average diameter from 30 microns to 2000 microns.

2. The injectable filler composition that can be applied to humans who suffer from a collagen sensitivity that will stimulate collagen production of claim 1, wherein the thickening agent is a composition that comprises:
from about 1.5% to about 3.0% of a carboxymethyl cellulose;
From about 0.05% to about 1.0% of a 1,4 butanediol diglycidyl ether;
from about 0.3% to about 0.6% of an ascorbic acid;
from about 0.2% to about 0.6% of a polysorbate;
from about 0.1% to about 0.2% of a methyl paraben;
0.3% of a lidocaine;
from about 0.01% to about 0.5% of a silica;
50% of a Lactated Ringer's Solution; and
from about 43.8% to about 47.54% of a sterile water solution for injection.

3. The injectable filler composition that can be applied to humans who suffer from a collagen sensitivity that will stimulate collagen production of claim 1, wherein the thickening agent is biodegradable when implanted subcutaneously in a human to provide a temporary scaffold for collagen growth between silicone oil droplets.
